(19) United States
(12) Patent Application Publication
(10) Pub. No.: US 2015/0076011 A1
(43) Pub. Date: Mar. 19, 2015

(54) LIQUID INHALATIONAL ANESTHETIC CONTAINER

(71) Applicant: PORTABLE ANESTHESIA SOLUTIONS LLC, Chesterfield, MO (US)

(72) Inventor: Douglas Steven Pernikoff, Glencoe, MO (US)

(73) Assignee: PORTABLE ANESTHESIA SOLUTIONS LLC, Chesterfield, MO (US)

(21) Appl. No.: 14/389,486

(22) PCT Filed: Apr. 1, 2013
(86) PCT No.: PCT/US2013/034863

§ 371 Date: Sep. 30, 2014

Related U.S. Application Data
(60) Provisional application No. 61/686,080, filed on Mar. 30, 2012.

(51) Int. Cl. A61M 16/18; B65D 25/42; B65D 25/14

(52) U.S. Cl. CPC A61M 16/183; B65D 25/14; B65D 25/42; A61M 2250/00; A61M 2202/04
USPC 206/205; 206/438

(57) ABSTRACT

A pliable container for a liquid inhalational anesthetic is disclosed. In various configurations, a pliable container comprises a pliable container body, a threaded spout, and a liquid inhalational anesthetic. The pliable container body can comprise a laminate, such as a laminate comprising at least one aluminum layer and at least one polymer layer. In some configurations, the liquid inhalational anesthetic can be in contact with a polymer layer. In some configurations, the liquid inhalational anesthetic can be in contact with an aluminum layer. In various configurations, a pliable container can further comprise a key collar, a cap, and a seal.

### PRIORITY STATEMENT

This application claims the benefit of and priority to U.S. Provisional Application No. 61/686,080, filed on Mar. 30, 2012, which is incorporated herein by reference in its entirely.

The present invention is in the field of packaging for inhalational anesthetics.

Previously disclosed liquid inhalational anesthetic containers are rigid and non-pliable. For example, many inhalational anesthetics, including liquid inhalational anesthetics, are typically supplied to medical institutions and medical practitioners in glass bottles. Such bottles are connected directly to an anesthesia machine or vaporizer, or indirectly through an intermediate adapter. In addition, inhalational anesthetic bottles for anesthetics such as sevoflurane, desflurane, isoflurane, halothane, methoxyflurane, ethrane and ether are usually provided with a key collar which includes protrusions and/or notches. The receiving device to which the bottle attaches can include a complementary key, such that each type of inhalational anesthetic has a unique key to minimize the likelihood of attaching a bottle comprising one anesthetic to a connection intended for a different anesthetic. FIG. 1 displays an example of an anesthetic bottle and adapter. FIG. 1A shows a prior art anesthetic bottle 10 separate from a vaporizer filler adapter 90. An anesthetic bottle connected with an adapter is shown in FIG. 1B. In FIG. 10A, anesthetic bottle 10 includes a spout 11 having a rim of standard outer diameter (22 mm. under ISO standards), screw thread 20, key collar 13 having protrusions 14. An adapter which receives the anesthetic bottle includes tube 91, connector 92, key 93 having slots 94. When the anesthetic bottled and adapter are properly connected (FIG. 1B), the protrusions 14 of the bottle's key collar match with the slots 94 of the adapter's key (see, e.g., U.S. Patent Application Publication 2008/0308179 of Danelsen; U.S. Patent Application Publication 2004/0206417 of Falligant; PCT application publication WO 95/13972 of De Rubeis et al). The key collars and adapter keys can further be color coded, for example following industry standards such as those set by the ISO.

FIG. 1 depicts an example of a prior art anesthetic glass bottle and a vaporizer filler adapter. As illustrated in FIG. 1A, glass bottle 10 includes spout with circular lip 11, screw threads 12, key collar 13, and key protrusions 14. The key collar can include at least 2 protrusions, wherein the key collar has protrusions patterned to serve as a key unique to a species of liquid inhalation anesthetic. A vaporizer filler adaptor 90 includes a connector 92, and a key 93. The key 93 includes slots 94 complementary to the patterned protrusions on key collar 13. The pattern of protrusions and the complementary slots are configured to be unique for each species of liquid inhalation anesthetic. When the bottle and the adaptor are connected as shown in FIG. 1B, the protrusions 14 and slots 94 match up to reduce the possibility of improper connection between a bottled liquid inhalational anesthetic and a filler adapter.

However, standard anesthetic bottles, being made from nonpliable glass, are subject to breakage and shattering. Furthermore, glass containers can be considered heavy to transport, and some liquid inhalational anesthetics are believed to react with or corrode glass or components thereof. For example, the fluorocarbon anesthetic sevoflurane as supplied by commercial suppliers in glass containers has been found to include contaminants such as hydrogen fluoride, speculated glass, and Lewis acids (Baker, M. T., Anesthesia & Analgesia 108, 1725-1726, 2009; Kharasch, E. D., et al., Anesthesia & Analgesia 108, 1796-1802, 2009; O'Neill, B., et al., Anesthesia 62, 421, 2007; U.S. Patent Application Publication 2010/0218762 of Jiang; U.S. Pat. No. 5,990,176 to Bieniarz et al.).

Alternatives to glass as packaging material for liquid inhalational anesthetics have been proposed. For example, U.S. Pat. No. 4,250,334 teaches the use of “Kel-F” plastic for a container for holding the fluorocarbon anesthetic sevoflurane, “Kel-F” is understood to be the trade name for chlorotrifluoroethylene, U.S. Pat. No. 5,679,576 teaches the use of a container lined with polytetrafluoroethylene (PTFE), for holding the fluorocarbon anesthetic sevoflurane. U.S. Pat. No. 5,505,236, teaches the use of a plastic container with an inhalational anesthetic. While not specifically naming the inhalational anesthetic, it is believed that the commercial embodiment of the system shown in the '236 patent has been used with the fluorocarbon anesthetic sevoflurane. Additional patents have taught the use of particular types of plastic for containers to hold sevoflurane. See, e.g., U.S. Pat. Nos. 6,074,668 (polyethylene napthalate), 6,083,514 (polymethylpentene) and 6,162,443 (polypropylene, polyethylene and ionomeric resins). In addition, containers for inhalational anesthetics made of certain plastics have been proposed. Cf. WO 99/34762, U.S. Pat. Nos. 6,074,668 to Flament-Garcia et al., 6,162,443 to Flament-Garcia et al., and U.S. Patent Application Publication No. U.S. 2001/0000729 A1 of Flament-Garcia et al. While plastic containers are less likely to break than glass containers, rigid plastic containers are still susceptible to breakage under common use conditions.

Containers for sevoflurane have also been made of stainless steel. For example, U.S. Pat. No. 5,990,176 describes a container made of glass, plastic or stainless steel for holding sevoflurane. U.S. Pat. No. 6,008,273 describes an epoxy resin for coating the inside of a metal container for use as a food or beverage container. U.S. Patent application publication 20110284418 of Rudzinski et al. discloses an aluminum container for inhalational anesthetics. However, the aluminum container is not described as pliable, deformable, or flexible. U.S. Patent application publication 20100286648 of Freed discloses a receptacle

### SUMMARY

The present inventor has developed pliable containers for liquid inhalational anesthetics. In various embodiments, the packaging can comprise a pliable material. In various embodiments, a pliable container of the present teachings can comprise or consist of: a pliable container body; a threaded spout; and a liquid inhalational anesthetic. In various embodiments, a pliable container body can comprise or consist of a laminate such as a multi-layer film.

In various embodiments, a laminate of a container body of the present teachings can comprise or consist of at least one layer of aluminum such as aluminum foil, and at least one layer of a polymer which is in contact with a liquid inhalational anesthetic. In various embodiments, a laminate of a container body of the present teachings can comprise or consist of at least one layer of a polymer, and at least one layer of aluminum such as aluminum foil which is in contact with a liquid inhalational anesthetic.

In various embodiments, a laminate of a container body of the present teachings can comprise or consist of at least one layer of aluminum such as aluminum foil, and two or more layers of polymer(s), including a polymer layer which is in contact with a liquid inhalational anesthetic. In various embodiments, a laminate of a container body of the present teachings can comprise or consist of two or more layers of polymer(s), and at least one layer of aluminum such as aluminum foil which is in contact with a liquid inhalational anesthetic. In. these embodiments, the polymers can be the same or different.

In various embodiments of the present teachings, a liquid inhalational anesthetic of the present teachings can be any liquid inhalational anesthetic known to skilled artisans. In some configurations, an inhalational anesthetic can be a fluorocarbon anesthetic, such as, without limitation, sevoflurane, desflurane, isoflurane, halothane or methoxyflurane. In some configurations, a liquid inhalational anesthetic can be selected from the group consisting of sevoflurane, desflurane, isoflurane, halothane, methoxyflurane, ethrane and ether.

In various embodiments, a pliable container of the present teachings can further comprise a key collar. Key collars of the present teachings are well known to skilled artisans, and follow industry standards for anesthetic bottles such as, without limitation, standards established by the ISO.

In various embodiments, a spout of a container of the present teachings can have dimensions that accord with industry standards, such as, without limitation, ISO standards for liquid inhalational anesthetic bottles, such as a cylindrical spout having a ˜29 mm outside diameter, and can be made of a plastic, such as a hard, non-deformable plastic such as, without limitation, high density polyethylene. In various configurations, the thread of a spout can be a screw thread. In various embodiments, a pliable container of the present teachings can further comprise a seal which covers the spout. The seal can comprise a plastic and/or a metal such as aluminum foil, and can function to maintain a liquid inhalational anesthetic within the container, and also to prevent evaporation. The seal can be maintained intact during storage, and can be detached or punctured to allow flow of the liquid inhalational anesthetic through the spout. In some aspects, the seal can be configured to be a removable seal.

A container body of a pliable container of the present teachings can be adapted, for holding a volume of an inhalational anesthetic, which can be, for example and without limitation from about 50 ml to about 250 ml, or greater, such as 50 ml, 100 ml, 150 ml, 200 ml, 240 ml, 250 ml, or greater.

The material of a container body of the present teaching can comprise any flexible material that includes a pliable polymer and/or a pliable metal sheet such as an aluminum sheet (aluminum foil). In various configurations, the material and its component layers can serve as a barrier to leakage or evaporation of a liquid inhalational anesthetic, and can be adapted from published references. For example, PCT application publication WO 03/097355 of Manabe discloses a plastic container for storing liquid medicines. The material disclosed in this publication is a multi-layer film having an a surface layer of ethylene-α-olefin copolymer, a pliable layer of an ethylene-α-olefin copolymer, a barrier layer of a cyclic olefin polymer and an ethylene-α-olefin copolymer, and a seal layer of an ethylene-α-olefin. In other configurations of the present teachings, a polymer layer can he a cyclic olefin co-polymer, or a cyclic olefin co-polymer (COC) and graft copolymer of ethylene and octene. In some configurations, a polymer layer of a pliable container body of the present teachings can be a layer of a polymer such as nylon, polyethylene or polypropylene. In various configurations, a polyethylene layer can be a linear low-density polyethylene (LLDPE). In various configurations, a polyethylene layer can be a biaxially-oriented polyethylene terephthalate (MYLAR® (Dupont Corp.)). In some configurations, the Mylar® can be metallized by vapor deposition to produce a thin film of an evaporated metal such as aluminum or gold. In some configurations, a first polymer layer, which can be in contact with a liquid inhalational anesthetic, can be LLDPE, while a second polymer layer can be nylon. In some configurations, a first layer can be biaxially-oriented polyethylene terephthalate and the second polymer layer can be nylon. In some configurations, a laminate can be trilaminate, and can comprise or consist of nylon sandwiched between aluminum and LLDPE. In some configurations, a laminate can comprise or consist of nylon sandwiched between aluminum and biaxially-oriented polyethylene terephthalate. In some configurations, a laminate can comprise or consist of alum mum sandwiched between nylon and LLDPE. In some configurations, a laminate can comprise or consist of aluminum sandwiched between nylon and biaxially-oriented polyethylene terephthalate. In some configurations, a laminate can comprise or consist of LLDPE sandwiched between, nylon and aluminum. In some configurations, a laminate can comprise or consist of biaxially-oriented polyethylene terephthalate sandwiched between nylon and aluminum.

In some embodiments, a pliable container of the present teachings can further include a cap. In various configurations, a cap can comprise or consist of a polymer selected from the group consisting, of high density polyethylene, LLDPE and biaxially-oriented polyethylene terephthalate. In various configurations, a cap can comprise or consist of aluminum. In various configurations, a cap can comprise or consist of a combination of a polymer and aluminum.

Embodiments of the present teachings include methods of anesthetizing a subject. In various configurations, these methods include fluidly connecting a container disclosed herein to an anesthesia machine; fluidly connecting the anesthesia machine to a subject; and administering an anesthetically effective amount of the inhalational anesthetic to the subject. In various configurations, a connection between a container and an anesthesia machine can be direct or indirect, e.g., through an adapter. In various configurations, a subject can be a human or an animal and the anesthetic can be any liquid inhalational anesthetic, such as a fluorocarbon anesthetic. In various configurations, the anesthetic can be selected from the group consisting of sevoflurane, desflurane, isoflurane, halothane, methoxyflurane, ethrane and ether, or selected from the group consisting of sevoflurane, desflurane, isoflurane, halothane and methoxyflurane. In various configurations, a container can further comprise a key collar, and can be connected to a machine or adapter comprising a complementary key. In various configurations, a key collar and key can correspond to ISO standards.

Embodiments of the present teachings include methods of delivering a liquid inhalational anesthetic to an anesthetic vaporizer reservoir. In various configurations, these methods comprise attaching a container disclosed herein comprising a liquid inhalational anesthetic to a key fill adaptor, and transferring the liquid anesthetic to the vaporizer reservoir. A liquid inhalational anesthetic of these embodiments can be any liquid inhalational anesthetic disclosed herein, such as, without limitation, sevoflurane, desflurane, isoflurane, halothane, methoxyflurane, ethrane or ether. In some configurations, a pliable container of these embodiments can comprise a key collar, such as a key collar which meets ISO standards for an inhalational anesthetic bottle key collar. In some configurations, a device for receiving a pliable container can comprise a key fill adaptor which comprises a complementary key fitting.

Embodiments of the present teachings also include various kits. A kit of the present teaching, in various configurations, can comprise a pliable container for a liquid inhalational anesthetic, wherein the pliable container comprises a pliable container body and a threaded spout; and at least one liquid inhalational anesthetic. In various configurations, a pliable container can comprise a laminate as described herein.

Embodiments of the present teachings include use of a material comprising aluminum foil, a first polymer, and a second polymer in the manufacture of a pliable container for storing a liquid inhalational anesthetic.

The present teachings include, without limitation, the following aspects.

1. A pliable container for a liquid inhalational anesthetic, comprising:

a pliable container body;

a threaded spout; and

a liquid inhalational anesthetic.

2. A pliable container in accordance with aspect 1, wherein the pliable container body comprises a laminate comprising an aluminum layer and a first polymer layer.
3. A pliable container in accordance with aspect 2, wherein the liquid inhalational anesthetic is in contact with the first polymer layer.
4. A pliable container in accordance with aspect 2, wherein the liquid inhalational anesthetic is in contact with the aluminum layer.
5. A pliable container in accordance with any one of aspects 1-4, wherein the liquid inhalational anesthetic is a fluorocarbon anesthetic.
6. A pliable container in accordance with any one of aspects 1-4, wherein the liquid inhalational anesthetic is selected from the group consisting of sevoflurane, desflurane, isoflurane, halothane, methoxyflurane, ethrane and ether.
7. A pliable container in accordance with any one of aspects 1-4, wherein the inhalational anesthetic is sevoflurane.
8. A pliable container in accordance with any one of aspects 1-4, wherein the inhalational anesthetic is desflurane.
9. A pliable container in accordance with any one of aspects 1-4, wherein the inhalational anesthetic is isoflurane.
10. A pliable container in accordance with any one of aspects 1-4, wherein the inhalational anesthetic is halothane.
11. A pliable container in accordance with any one of aspects 1-4, wherein the inhalational anesthetic is methoxyflurane.
12. A pliable container in accordance with any one of aspects 1-4, wherein the inhalational anesthetic is ethrane.
13. A pliable container in accordance with any one of aspects 1-4, wherein the inhalational anesthetic is ether.
14. A liable container in accordance with any one of aspects 1-4, further comprising a key collar.
15. A pliable container in accordance with any one of aspects 1-4, further comprising a key collar corresponding to an ISO standard key collar for an inhalational anesthetic bottle.
16. A pliable container in accordance with any one of aspects 1-4, further comprising a seal which covers the threaded spout.
17. A pliable container in accordance with aspect 16, wherein the seal is a detachable seal.
18. A pliable container in accordance with any one of aspects 1-4, wherein the threaded spout comprises a screw thread.
19. A pliable container in accordance with any one of aspects 2-4, wherein the first polymer layer comprises cyclic olefin co-polymer.
20. A pliable container in accordance with any one of aspects 2-4, wherein the first polymer

layer comprises cyclic olefin co-polymer (COC) and graft copolymer of ethylene and octene.

21. A pliable container in accordance with any one of aspects 2-4, wherein the first polymer layer comprises nylon.
22. A pliable container in accordance with any one of aspects 2-4, wherein the first polymer layer comprises a polyethylene.
23. A pliable container in accordance with aspect 22, wherein the polyethylene is linear low-density polyethylene (LLDPE).
24. A pliable container in accordance with aspect 22, wherein the polyethylene is biaxially-oriented polyethylene terephthalate.
25. A pliable container in accordance with any one of aspects 2-4, wherein the first polymer layer comprises polypropylene.
26. A pliable container in accordance with any one of aspects 2-4, wherein the laminate further comprises a second polymer layer.
27. A pliable container in accordance with aspect 26, wherein the second polymer layer comprises cyclic olefin co-polymer.
28. A pliable container in accordance with aspect 26, wherein the second polymer layer comprises cyclic olefin co-polymer and graft co-polymer of ethylene and octene.
29. A pliable container in accordance with aspect 26, wherein the second polymer layer comprises nylon.
30. A pliable container in accordance with aspect 26, wherein the second polymer layer comprises a polyethylene.
31. A pliable container in accordance with aspect 30, wherein the polyethylene of the second polymer layer is linear low-density polyethylene (LLDPE).
32. A pliable container in accordance with aspect 30, wherein the polyethylene of the second polymer layer is biaxially-oriented polyethylene terephthalate.
33. A pliable container in accordance with aspect 26, wherein the second polymer layer comprises polypropylene.
34. A pliable container in accordance with aspect 26, wherein the first polymer layer comprises LLDPE and the second polymer layer comprises nylon.
35. A pliable container in accordance with aspect 26, wherein the first polymer layer comprises biaxially-oriented polyethylene terephthalate and the second polymer layer comprises nylon.
36. A pliable container in accordance with aspect 26, wherein the laminate comprises nylon sandwiched between aluminum and LLDPE.
37. A pliable container in accordance with aspect 26, wherein the laminate comprises nylon sandwiched between aluminum and biaxially-oriented polyethylene terephthalate.
38. A pliable container in accordance with one of aspects 1-4, further comprising a cap.
39. A pliable container in accordance with aspect 38, wherein the cap consists of a polymer selected from the group consisting of high density polyethylene, LLDPE and biaxially-oriented polyethylene terephthalate.
40. A method of anesthetizing a subject, comprising:

fluidly connecting a container of any one of aspects 1-4 to an anesthesia machine;

fluidly connecting the anesthesia machine to a subject; and

administering an anesthetically effective amount of the inhalational anesthetic to the subject.

41. A method of anesthetizing a subject in accordance with aspect 40, wherein the subject is a human.
42. A method of anesthetizing a subject in accordance with aspect 40, wherein the subject is an animal.
43. A method of anesthetizing a subject in accordance with aspect 40, wherein the liquid inhalational anesthetic is a fluorocarbon anesthetic.
44. A method of anesthetizing a subject in accordance with aspect 40, wherein the liquid inhalational anesthetic is selected from the group consisting of sevoflurane, desflurane, isoflurane, halothane, methoxyflurane, ethrane and ether.
45. A method of anesthetizing a subject in accordance with aspect 40, wherein the inhalational anesthetic is selected from the group consisting of sevoflurane, desflurane, isoflurane, halothane and methoxyflurane.
46. A method of anesthetizing a subject in accordance with aspect 40, wherein the container further comprises a key collar.
47. A method of anesthetizing a subject in accordance with aspect 40, wherein the container further comprises a key collar corresponding to an ISO standard key collar for an inhalational anesthetic bottle.
48. A method of delivering a liquid inhalational anesthetic to an anesthetic vaporizer reservoir, comprising:

attaching a container of any one of aspects 1-4 to a key fill adaptor; and

transferring the liquid anesthetic to the vaporizer reservoir.

49. A. method in accordance with aspect 48, wherein the liquid inhalational anesthetic is selected from the group consisting of sevoflurane, desflurane, isoflurane, halothane, methoxyflurane, ethrane and ether.
50. A method of anesthetizing a subject in accordance with aspect 48, wherein the inhalational anesthetic is selected from the group consisting of sevoflurane, desflurane, isoflurane, halothane and methoxyflurane.
51. A method of anesthetizing a subject in accordance with aspect 48, wherein the container further comprises a key collar.
52. A method of anesthetizing a subject in accordance with aspect 48, wherein the container further comprises a key collar corresponding to an ISO standard key collar for an inhalational anesthetic bottle.
53. A method of anesthetizing a subject in accordance with aspect 48, wherein the container comprises an anesthetic-specific key collar and the key fill adaptor comprises a complementary key fitting.
54. A kit comprising:

a pliable container for a liquid inhalational anesthetic, wherein the pliable container comprises a pliable container body and a threaded spout; and

at least one liquid inhalational anesthetic.

55. A kit in accordance with aspect 54, wherein the pliable container comprises a laminate comprising an aluminum layer and a first polymer layer.
56. A kit in accordance with aspect 54, wherein the first polymer layer is in contact with the interior of the container.
57. A kit in accordance with aspect 54, wherein the aluminum layer is in contact with the interior of the container.
58. A kit in accordance with any one of aspects 54-57, wherein the at least one liquid inhalational anesthetic is a fluorocarbon anesthetic.
59. A kit in accordance with any one of aspects 54-57, wherein the at least one liquid inhalational anesthetic is selected from the group consisting of sevoflurane, desflurane, isoflurane, halothane, methoxyflurane, ethrane and ether.
60. A kit in accordance with any one of aspects 54-57, wherein the at least one liquid inhalational anesthetic is sevoflurane.
61. A kit in accordance with any one of aspects 54-57, wherein the at least one liquid inhalational anesthetic is desflurane.
62. A kit in accordance with any one of aspects 54-57, wherein the at least one liquid inhalational anesthetic is isoflurane.
63. A kit in accordance with any one of aspects 54-57, wherein the at least one liquid inhalational anesthetic is halothane.
64. A kit in accordance with any one of aspects 54-57, wherein the at least one liquid inhalational anesthetic is methoxyflurane.
65. A kit in accordance with any one of aspects 54-57, wherein the at least one liquid inhalational anesthetic is ethrane.
66. A kit in accordance with any one of aspects 54-57, wherein the at least one liquid inhalational anesthetic is ether.
67. A kit in accordance with any one of aspects 54-57, further comprising at least one key collar.
68. A kit in accordance with any one of aspects 54-57, further comprising at least one key collar corresponding to an ISO standard key collar for an inhalational anesthetic bottle.
69. A kit in accordance with any one of aspects 54-57, wherein the pliable container further comprises a seal which covers the threaded spout.
70. A kit in accordance with aspect 69, wherein the seal is a detachable seal.
71. A kit in accordance with any one of aspects 54-57, wherein the threaded spout comprises a screw thread.
72. A kit in accordance with any one of aspects 54-57, wherein the first polymer layer comprises cyclic olefin co-polymer.
73. A kit in accordance with any one of aspects 54-57, wherein the first polymer layer comprises cyclic olefin co-polymer (COC) and graft copolymer of ethylene and octene.
74. A kit in accordance with any one of aspects 54-57, wherein the first polymer layer comprises nylon.
75. A kit in accordance with any one of aspects 54-57, wherein the first polymer layer comprises a polyethylene.
76. A kit in accordance with aspect 75, wherein the polyethylene is LLDPE.
77. A kit in accordance with aspect 75, wherein the polyethylene is biaxially-oriented polyethylene terephthalate.
78. A kit in accordance with any one of aspects 54-57, wherein the first polymer layer comprises polypropylene.
79. A kit in accordance with any one of aspects 54-57, wherein the laminate further comprises a second polymer layer.
80. A kit in accordance with aspect 79, wherein the second polymer layer comprises cyclic olefin co-polymer.
81. A kit in accordance with aspect 79, wherein the second polymer layer comprises cyclic olefin co-polymer and graft, co-polymer of ethylene and octene.
82. A kit in accordance with aspect 79, wherein the second polymer layer comprises nylon.
83. A kit in accordance with aspect 79, wherein the second polymer layer comprises a polyethylene.
84. A kit in accordance with aspect 83, wherein the polyethylene of the second polymer layer is linear low-density polyethylene (LLDPE).
85. A kit in accordance with aspect 83, wherein the polyethylene of the second polymer layer is biaxially-oriented polyethylene terephthalate.
86. A kit in accordance with aspect 79, wherein the second polymer layer comprises polypropylene.
87. A kit in accordance with aspect 79, wherein the first polymer layer comprises a polyethylene and the second polymer layer comprises nylon.
88. A kit in accordance with aspect 79, wherein the first polymer layer is a polyethylene layer, the second polymer layer is a nylon layer, and the laminate comprises the nylon layer sandwiched between the aluminum layer and the polyethylene layer.
89. A kit in accordance with aspect 79, wherein the first polymer layer is a polyethylene layer, the second polymer layer is a nylon layer, and the laminate comprises the aluminum layer sandwiched between the nylon layer and the polyethylene layer.
90. Use of a material comprising aluminum foil, a first polymer, and a second polymer in the manufacture of a pliable container for storing a liquid inhalational anesthetic.

### BRIEF DESCRIPTION OF THE DRAWINGS

FIG. 1 illustrates an example of a prior art liquid inhalational anesthetic glass bottle and adapter, wherein FIG. 1A illustrates a glass bottle separate from an adapter, and FIG. 1B illustrates a glass bottle connected to an adapter.

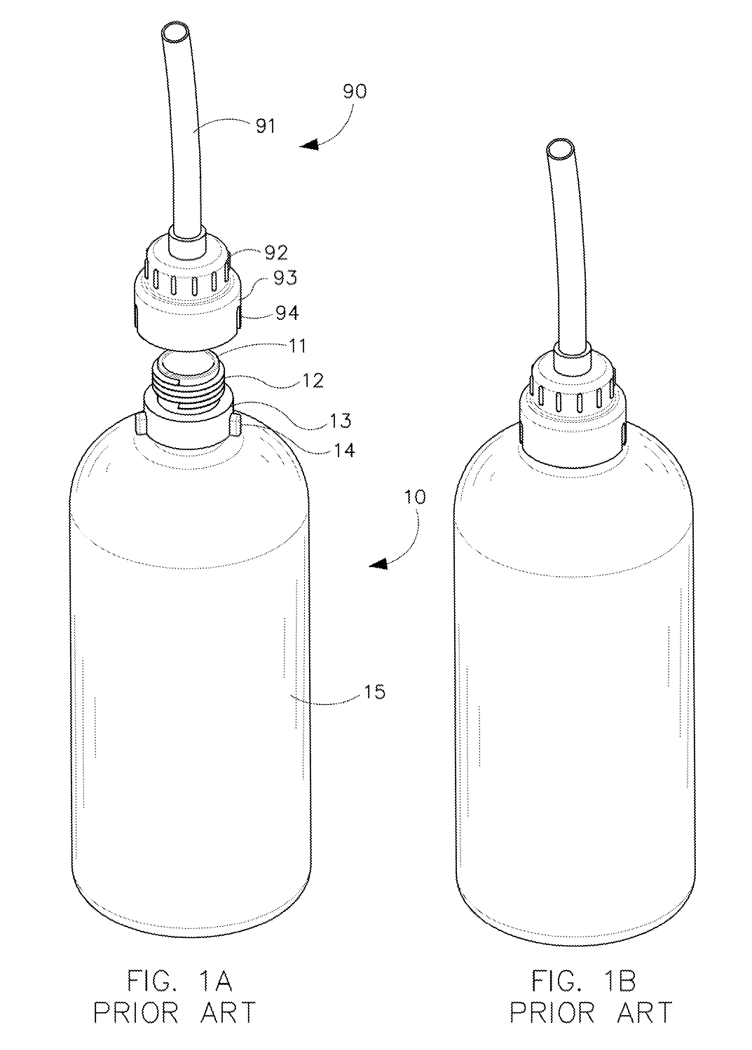

FIG. 2 illustrates a configuration of the present teachings resembling a toothpaste tube. FIG. 2A illustrates pliable container separate from an adapter, and FIG. 2B illustrates a pliable container connected to an adapter.

FIG. 3 illustrates a configuration of the present teachings in the shape resembling a flask. FIG. 3A illustrates a pliable container separate from an adapter, and FIG. 3B illustrates a pliable container connected to an adapter.

FIG. 4 illustrates a configuration of the present teachings in the shape resembling a flask. FIG. 4A. illustrates a pliable container separate from an. adapter, and FIG. 4B illustrates a pliable container connected to an adapter.

FIG. 5 illustrates a configuration of the present teachings in the shape resembling a disposable juice container. FIG. 4A illustrates a pliable container separate from an adapter, and FIG. 4B illustrates a pliable container connected to an adapter.

### DETAILED DESCRIPTION

The present inventor has developed pliable containers for liquid inhalational anesthetics. In various embodiments, the packaging can comprise a pliable material. In various embodiments, a pliable container of the present teachings can comprise or consist of: a pliable container body; a threaded spout; and a liquid inhalational anesthetic. In various embodiments, a pliable container body can comprise or consist of a laminate such as a multi-layer film.

In various embodiments, a laminate of a container body of the present teachings can comprise or consist of at least one layer of aluminum such as aluminum foil, and at least one layer of a polymer wherein the polymer layer is in contact with a liquid inhalational anesthetic. In various embodiments, a laminate of a container body of the present teachings can comprise or consist of at least one layer of a polymer, and at least one layer of aluminum such as aluminum foil wherein the aluminum layer is in contact with a liquid inhalational anesthetic. In various configurations, a polymer of a laminate of the present disclosure can be any polymer which can form a flexible material. In some configurations, a polymer can also serve as a flexible barrier to liquid anesthetic flow and evaporation. Non-limiting examples of polymers, films and laminates which can be produced and used in a container body of the present teachings are disclosed in publications such as U.S. Patent Application Publication 20100218762 of Jiang; U.S. Patent Application Publication 20070243293 of Janssen et al.; U.S. Patent Application Publication 20080071046 of Leclerc et al; U.S. Pat. No. 5,837,787 to Harrington; U.S. Patent Application Publication 20080299370 of Briggs; U.S. Patent Application Publication 20070026251 of Umana; U.S. Patent Application Publication 20070088145 of Mgaya et al., U.S. Patent Application Publication 20080099141 of Booth et al, U.S. Pat. No. 7,384,674 to Andersson et al., U.S. Patent Application Publication 20120238955 of Wyss; U.S. Pat. No. 8,129,031 to Ling et al; and U.S. Patent Application Publication 20110064955 of Bertolino et al.

A container body of the present teachings can comprise or consist of, in non-limiting example, a polyethylene layer that can be a biaxially-oriented polyethylene terephthalate (Mylar®) film. In various configurations, a flexible container body of the present teachings can comprise at least one layer of a polymer such as a cyclic olefin co-polymer, for example TOPAS® 8007 (Polyplastics Co., Ltd.) or Exact® Plastomers (EXACT 5061, ExxonMobil Chemical). In various configurations, a container body of the present teachings can have, in non-limiting examples, a thickness of 0.002, 0.0048, 0.005, 0.0075, 0.010 or 0.014 inch. In some configurations, a container body can comprise 5 mil Mylar®. In some configurations, 5 mil thick Mylar® can be used having a middle layer of aluminum (0.00035″). In some configurations, an aluminum layer can be from 0.00035″ to 0.078″ thick, such as, for example, about 0.001″ thick. In some configurations, a pliable container body can be made from a 0.35 mil (9 μm) thick Mylar® film sandwiched between two layers of 0.18 mil (4.5 μm) thick aluminum foil that are bonded together. In some configurations, a pliable container body can be made of aluminized Mylar® film that can be laminated with a layer of polyethylene.

Polymers and metal foils and laminate materials can be formed using methods well known to skilled artisans. Furthermore, materials can be shaped and containers assembled using methods known to skilled artisans.

Key collars can correspond to industry standards, and furthermore can be color-coded in accordance with industry standards, for example yellow for sevoflurane, purple for isoflurane, red for halothane, and blue for desflurane.

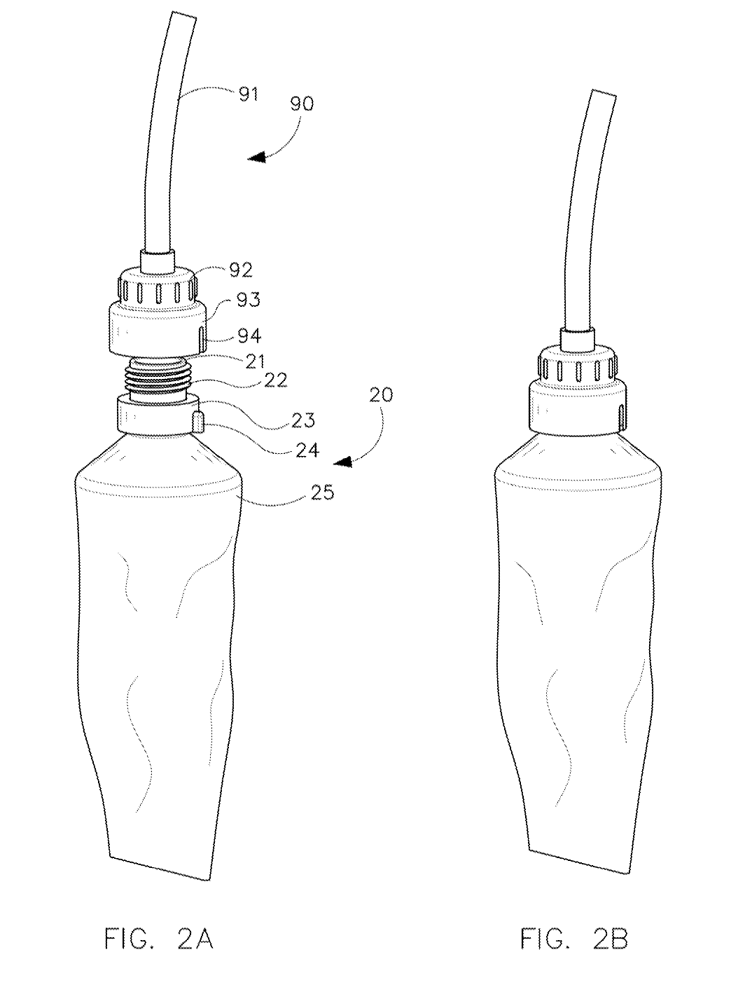

FIG. 2 depicts an example of a container of the present teachings, and a vaporizer filler adapter. As illustrated in FIG. 2A, a configuration resembling a toothpaste tube 20 includes spout with circular lip 21, screw threads 22, key collar 23, and key protrusions 24. The key collar can include at least 2 protrusions, wherein the key collar has protrusions patterned to serve as a key unique to a species of liquid inhalation anesthetic. A vaporizer filler adaptor 90 includes a connector 92, and a key 93. The key 93 includes slots 94 complementary to the patterned protrusions on key collar 23. The pattern of protrusions and the complementary slots are configured to be unique for each species of liquid inhalation anesthetic. When the bottle and the adaptor are connected as shown in FIG. 2B, the protrusions 24 and slots 94 match up to reduce the possibility of improper connection between a bottled liquid inhalational anesthetic and a filler adapter. In various aspects, a container of this configuration can have either a tapered or a pinched base.

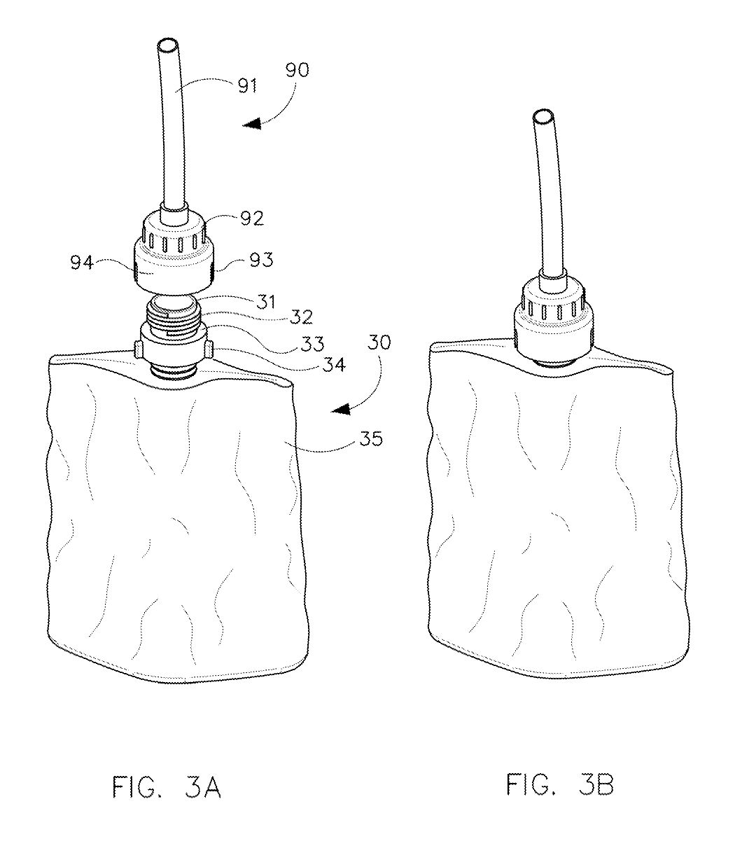

FIG. 3 depicts an example of a container of the present teachings, and a vaporizer filler adapter. As illustrated in FIG. 3A, a configuration resembling a flask 30 includes spout with circular lip 31, screw threads 32, key collar 33, and key protrusions 34. The key collar can include at least 2 protrusions, wherein the key collar has protrusions patterned to serve as a key unique to a species of liquid inhalation anesthetic. A vaporizer filler adaptor 90 includes a connector 92, and a key 93. The key 93 includes slots 94 complementary to the patterned protrusions on key collar 33. The pattern of protrusions and the complementary slots are configured to be unique for each species of liquid inhalation anesthetic. When the bottle and the adaptor are connected as shown in FIG. 3B, the protrusions 34 and slots 94 match up to reduce the possibility of improper connection between a bottled liquid inhalational anesthetic and a filler adapter. In various aspects, a container of this configuration can include a gusset, at the base to allow expansion upon introduction of a liquid inhalational anesthetic, or contraction during draining of the anesthetic.

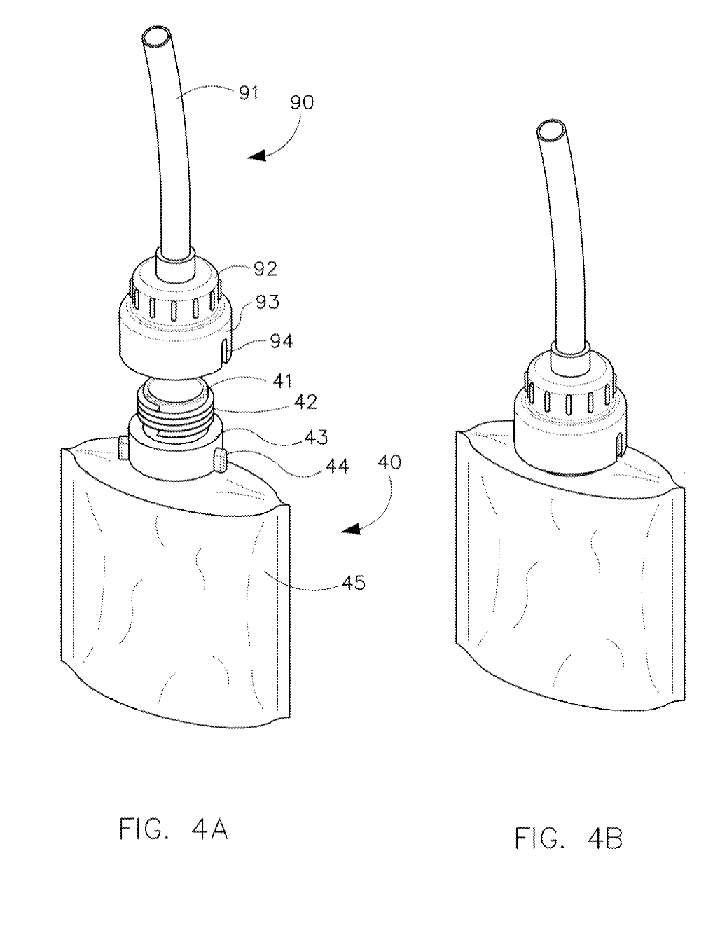

FIG. 4 depicts an example of a container of the present teachings, and a vaporizer filler adapter. As illustrated in FIG. 4A, a flask-like configuration 40 includes spout with circular lip 41, screw threads 42, key collar 43, and key protrusions 44. The key collar can include at least 2 protrusions, wherein the key collar has protrusions patterned to serve as a key unique to a species of liquid inhalation anesthetic. A vaporizer filler adaptor 90 includes a connector 92. and a key 93. The key 93 includes slots 94 complementary to the patterned protrusions on key collar 43. The pattern of protrusions and the complementary slots are configured to be unique for each species of liquid inhalation anesthetic. When the bottle and the adaptor are connected as shown in FIG. 4B, the protrusions 44 and slots 94 match up to reduce the possibility of improper connection between a bottled liquid inhalational anesthetic and a filler adapter. In various aspects, a container of this configuration can include a gusset as in FIG. 3.

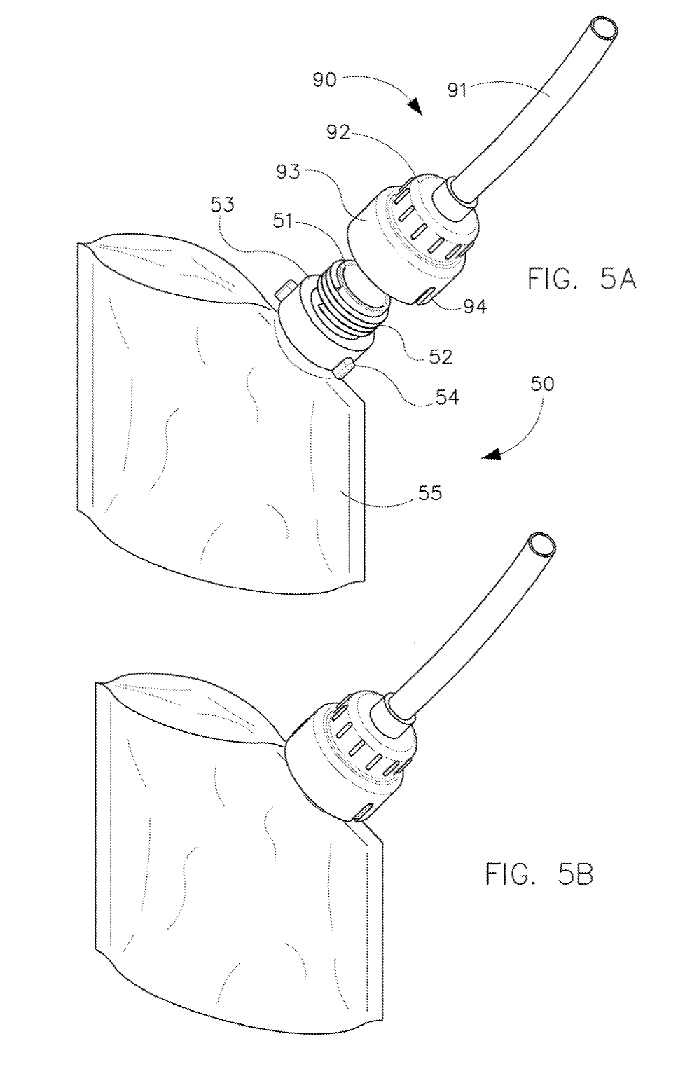

FIG. 5 depicts an example of a container of the present teachings, and a vaporizer filler adapter. As illustrated in FIG. 5A, a configuration resembling a disposable juice container 50 includes spout with circular lip 51, screw threads 52, key collar 53, and key protrusions 54. The key collar can include at least 2 protrusions, wherein the key collar has protrusions patterned to serve as a key unique to a species of liquid inhalation anesthetic. A vaporizer filler adaptor 90 includes a connector 92, and a key 93. The key 93 includes slots 94 complementary to the patterned protrusions on key collar 53. The pattern of protrusions and the complementary slots are configured to be unique for each species of liquid inhalation anesthetic. When the bottle and the adaptor are connected as shown in FIG. 5B, the protrusions 54 and slots 94 match up to reduce the possibility of improper connection between a bottled liquid inhalational anesthetic and a filler adapter.

All references cited are incorporated herein by reference, each in its entirety.

What is claimed is:

1. A pliable container for a liquid inhalational anesthetic, comprising:
a pliable container body;
a threaded spout; and
a liquid inhalational fluorocarbon anesthetic.

2. A pliable container in accordance with claim 1, wherein the pliable container body comprises a laminate comprising an aluminum layer and a first polymer layer.

3. A pliable container in accordance with claim 2, wherein the liquid inhalational anesthetic is in contact with the first polymer layer.

4. A pliable container in accordance with claim 2, wherein the liquid inhalational anesthetic is in contact with the aluminum layer.

5. (canceled)

6. A pliable container in accordance with claim 1, wherein the liquid inhalational anesthetic is selected from the group consisting of sevoflurane, desflurane, isoflurane, halothane, and methoxyflurane, ethrane and ether.

7-13. (canceled)

14. A pliable container in accordance with claim 1, further comprising a key collar.

15. (canceled)

16. A pliable container in accordance with claim 1, further comprising a seal which covers the threaded spout.

17. (canceled)

18. A pliable container in accordance with claim 1, wherein the threaded spout comprises a screw thread.

19. A pliable container in accordance with claim 2, wherein the first polymer layer comprises a polymer selected from the group consisting of cyclic olefin co-polymer, graft copolymer of ethylene and octane, nylon, a polyethylene, and polypropylene.

20-22. (canceled)

23. A pliable container in accordance with claim 19, wherein the polyethylene is selected from the group consisting of linear low-density polyethylene (LLDPE) and biaxially-oriented polyethylene terephthalate.

24-25. (canceled)

26. A pliable container in accordance with claim 2, wherein the laminate further comprises a second polymer layer.

27. A pliable container in accordance with claim 26, wherein the second polymer layer comprises a polymer selected from the group consisting of cyclic olefin co-polymer, graft co-polymer of ethylene and octane, nylon, a polyethylene, polypropylene and nylon.

28-30. (canceled)

31. A pliable container in accordance with claim 27, wherein the polyethylene of the second polymer layer is selected from the group consisting of linear low-density polyethylene (LLDPE) and biaxially-oriented polyethylene terephthalate.

32-33. (canceled)

34. A pliable container in accordance with claim 26, wherein the first polymer layer comprises LLDPE and the second polymer layer comprises nylon.

35. A pliable container in accordance with claim 26, wherein the first polymer layer comprises biaxially-oriented polyethylene terephthalate and the second polymer layer comprises nylon.

36. A pliable container in accordance with claim 26, wherein the laminate comprises nylon sandwiched between aluminum and LLDPE.

37. A pliable container in accordance with claim 26, wherein the laminate comprises nylon sandwiched between aluminum and biaxially-oriented polyethylene terephthalate.

38-53. (canceled)

54. A kit comprising:
a pliable container for a liquid inhalational anesthetic, wherein the pliable container comprises a pliable container body and a threaded spout; and
at least one liquid inhalational fluorocarbon anesthetic.

55. A kit in accordance with claim 54, wherein the pliable container comprises a laminate comprising an aluminum layer and a first polymer layer.

56. A kit in accordance with claim 55, wherein the first polymer layer is in contact with the interior of the container.

57. A kit in accordance with claim 55, wherein the aluminum layer is in contact with the interior of the container.

58. A kit in accordance with claim 55, wherein the first polymer layer comprises a polymer selected from the group consisting of cyclic olefin co-polymer, graft copolymer of ethylene and octane, nylon, a polyethylene and polypropylene.

59-61. (canceled)

62. A kit in accordance with claim 58, wherein the polyethylene is selected from the group consisting of LLDPE and biaxially-oriented polyethylene terephthalate.

63. (canceled)

64. A kit in accordance with claim 55, wherein the laminate further comprises a second polymer layer.

65. A kit in accordance with claim 64, wherein the second polymer layer comprises a polymer selected from the group consisting of cyclic olefin co-polymer, graft co-polymer of ethylene and octane, nylon, a polyethylene and polypropylene.

66-68. (canceled)

69. A kit in accordance with claim 65, wherein the polyethylene is selected from the group consisting of linear low-density polyethylene (LLDPE) and biaxially-oriented polyethylene terephthalate.

70. (canceled)

71. A kit in accordance with claim 64, wherein the first polymer layer comprises a polyethylene and the second polymer layer comprises nylon.

72. A kit in accordance with claim 64, wherein the first polymer layer is a polyethylene layer, the second polymer layer is a nylon layer, and the laminate comprises the nylon layer sandwiched between the aluminum layer and the polyethylene layer.

73. A kit in accordance with claim 64, wherein the first polymer layer is a polyethylene layer, the second polymer layer is a nylon, layer, and the laminate comprises the aluminum layer sandwiched between the nylon layer and the polyethylene layer.

74. (canceled)

75. A pliable container in accordance with claim 54, wherein the at least one liquid inhalational fluorocarbon anesthetic is selected from the group consisting of sevoflurane, desflurane, isoflurane, halothane, and methoxyflurane.
